(19) United States
(12) Patent Application Publication
(10) Pub. No.: US 2026/0241085 A1
(43) Pub. Date: Aug. 20, 2026

(54) DIALYSIS SYSTEM AND METHODS

(71) Applicant: OUTSET MEDICAL, INC., San Jose, CA (US)

(72) Inventors: Michael Edward HOGARD, Odessa, FL (US); Michael ARAGON, Ft. Worth, TX (US); Dean HU, San Leandro, CA (US); Amy Elizabeth KERDOK, San Jose, CA (US); Elaine CAVALIER, Los Gatos, CA (US)

(21) Appl. No.: 19/643,101

(22) Filed: Apr. 9, 2026

Related U.S. Application Data
(62) Division of application No. 17/395,281, filed on Aug. 5, 2021, now abandoned.
(60) Provisional application No. 63/061,623, filed on Aug. 5, 2020.

(51) Int. Cl. A61M 1/16; A61M 1/36; A61M 39/28

(52) U.S. Cl. CPC A61M 1/1603; A61M 1/365; A61M 39/28; A61M 2205/3331; A61M 2205/3334

(57) ABSTRACT

Dialysis systems and methods are described which can include a number of features. The dialysis systems described can be to provide dialysis therapy to a patient in the comfort of their own home. The dialysis system can be configured to prepare purified water from a tap water source in real-time that is used for creating a dialysate solution. The dialysis systems described also include features that make it easy for a patient to self-administer therapy.

### CROSS REFERENCE TO RELATED APPLICATIONS

This application is a divisional of U.S. Application No. 17/395,281, filed August 5, 2021, titled “DIALYSIS SYSTEM AND METHODS”, which claims the benefit of priority of U.S. Provisional Application No. 63/061,623, filed August 5, 2020, which is herein incorporated by reference in its entirety.

### INCORPORATION BY REFERENCE

All publications and patent applications mentioned in this specification are herein incorporated by reference to the same extent as if each individual publication or patent application was specifically and individually indicated to be incorporated by reference.

### FIELD

This disclosure generally relates to dialysis systems. More specifically, this disclosure relates to the delivery of replacement fluid, normal saline, or other dilution fluid into a blood flow path. This disclosure is particularly adaptable to longer treatments, increasing user convenience through automation, and the reduction of nuisance alarms associated with automated saline delivery.

### BACKGROUND

There are, at present, hundreds of thousands of patients in the United States with end-stage renal disease. Most of those require dialysis to survive. Many patients receive dialysis treatment at a dialysis center, which can place a demanding, restrictive and tiring schedule on a patient. Patients who receive in-center dialysis typically must travel to the center at least three times a week and sit in a chair for 3 to 4 hours each time while toxins and excess fluids are filtered from their blood. After the treatment, the patient must wait for the needle site to stop bleeding and blood pressure to return to normal, which requires even more time taken away from other, more fulfilling activities in their daily lives. Moreover, in-center patients must follow an uncompromising schedule as a typical center treats three to five shifts of patients in the course of a day. As a result, many people who dialyze three times a week complain of feeling exhausted for at least a few hours after a session.

Many dialysis systems on the market require significant input and attention from technicians prior to, during, and after the dialysis therapy. Before therapy, the technicians are often required to manually install patient blood tubing sets onto the dialysis system, connect the tubing sets to the patient, and to the dialyzer, and manually prime the tubing sets to remove air from the tubing set before therapy. During therapy, the technicians are typically required to monitor venous pressure and fluid levels, and administer boluses of saline and/or heparin to the patient. After therapy, the technicians are often required to return blood in the tubing set to the patient and drain the dialysis system. The inefficiencies of most dialysis systems and the need for significant technician involvement in the process make it even more difficult for patients to receive dialysis therapy away from large treatment centers.

Given the demanding nature of in-center dialysis, many patients have turned to home dialysis as an option. Home dialysis provides the patient with scheduling flexibility as it permits the patient to choose treatment times to fit other activities, such as going to work or caring for a family member. Unfortunately, current dialysis systems are generally unsuitable for use in a patient’s home. One reason for this is that current systems are too large and bulky to fit within a typical home. Current dialysis systems are also energy-inefficient in that they use large amounts of energy to heat large amounts of water for proper use. Although some home dialysis systems are available, they generally are difficult to set up and use. As a result, most dialysis treatments for chronic patients are performed at dialysis centers.

Hemodialysis is also performed in the acute hospital setting, either for current dialysis patients who have been hospitalized, or for patients suffering from acute kidney injury. In these care settings, typically a hospital room, water of sufficient purity to create dialysate is not readily available. Therefore, hemodialysis machines in the acute setting rely on large quantities of pre-mixed dialysate, which are typically provided in large bags and are cumbersome for staff to handle. Alternatively, hemodialysis machines may be connected to a portable RO (reverse osmosis) machine, or other similar water purification device. This introduces another independent piece of equipment that must be managed, transported and disinfected.

### SUMMARY OF THE DISCLOSURE

A method of improving the performance of dialysis tubing that includes an arterial line and also includes a fluid delivery line connecting a fluid source to the dialysis tubing is provided, comprising the steps of occluding the arterial line of the dialysis tubing, opening or releasing a clamping mechanism that interfaces with the fluid delivery line, modulating a pump speed of a blood pump that interfaces with the dialysis tubing to increase a fluid pressure within the dialysis tubing, and delivering fluid into the dialysis tubing from the fluid source through the fluid delivery line.

In some embodiments, modulating the pump speed of the blood pump comprises increasing a flow rate of the blood pump from a first flow rate to a second flow rate.

In other embodiments, modulating the pump speed of the blood pump comprises decreasing a flow rate of the blood pump from a first flow rate to a second flow rate.

In one embodiment, modulating the pump speed of the blood pump comprises pulsing a flow rate of the blood pump.

In some examples, modulating the pump speed of the blood pump comprises decreasing a flow rate of the blood pump from approximately 320 ml/min to approximately 180 ml/min.

In another embodiment, the method further comprises automatically scheduling the occluding, opening, and modulating steps to occur periodically during dialysis therapy.

In one embodiment, a portion of the fluid delivery line remains occluded after opening or releasing the clamping mechanism due to prior extended clamping of the fluid delivery line.

In another embodiment, a portion of the fluid delivery line remains partially occluded after opening or releasing the clamping mechanism due to prior extended clamping of the fluid delivery line.

In one example, the method further comprises the steps of un-occluding the arterial line, closing or compressing the clamping mechanism that interfaces with the fluid delivery line, and initiating dialysis therapy.

A dialysis system is provided, comprising a fluid source, a dialysis tubing set that includes at least an arterial line, a blood pump portion, and a fluid delivery line that connects the fluid source to the dialysis tubing set, a blood pump configured to interface with the blood pump portion of the dialysis tubing set, a first clamping mechanism configured to interface with the arterial line, a second clamping mechanism configured to interface with the fluid delivery line; and an electronic controller configured to control operation of the blood pump, the first clamping mechanism, and the second clamping mechanism, wherein, during a priming sequence, the electronic controller is configured to close or occlude the first clamping mechanism, open or un-occlude the second clamping mechanism, modulate a pump speed of the blood pump to increase a fluid pressure within the dialysis tubing set and deliver fluid into the dialysis tubing from the fluid source through the fluid delivery line.

In one embodiment, the electronic controller is configured to modulate the pump speed of the blood pump by increasing a flow rate of the blood pump from a first flow rate to a second flow rate.

In another embodiment, the electronic controller is configured to modulate the pump speed of the blood pump by decreasing a flow rate of the blood pump from a first flow rate to a second flow rate.

In some examples, the electronic controller is configured to modulate the pump speed of the blood pump by pulsing a flow rate of the blood pump.

In one embodiment, the electronic controller is configured to modulate the pump speed of the blood pump comprises decreasing a flow rate of the blood pump from approximately 320 ml/min to approximately 180 ml/min.

In another embodiment, the controller is configured to automatically perform the close, open, and modulate steps periodically during dialysis therapy.

In some embodiments, a portion of the fluid delivery line remains occluded after opening or releasing the clamping mechanism due to prior extended clamping of the fluid delivery line.

In one embodiment, a portion of the fluid delivery line remains partially occluded after opening or releasing the clamping mechanism due to prior extended clamping of the fluid delivery line.

In some embodiments, the electronic controller is further configured to un-occlude the arterial line, close or compress the clamping mechanism that interfaces with the fluid delivery line, and initiate dialysis therapy.

A method of improving the performance of a dialyzer during dialysis therapy is provided, comprising the steps of initiating dialysis therapy, detecting a difference in pressure between a blood size of the dialyzer and a dialysate side of the dialyzer, if the difference in pressure exceeds a predetermined threshold, occluding the arterial line of the dialysis tubing, opening or releasing a clamping mechanism that interfaces with the fluid delivery line, modulating a pump speed of a blood pump that interfaces with the dialysis tubing to increase a fluid pressure within the dialysis tubing, and delivering fluid into the dialysis tubing from the fluid source through the fluid delivery line.

In some embodiments, modulating the pump speed of the blood pump comprises increasing a flow rate of the blood pump from a first flow rate to a second flow rate.

In other embodiments, modulating the pump speed of the blood pump comprises decreasing a flow rate of the blood pump from a first flow rate to a second flow rate.

In one embodiment, modulating the pump speed of the blood pump comprises pulsing a flow rate of the blood pump.

A method of returning blood to a patient after dialysis therapy with a dialysis system that includes a fluid delivery line connecting a fluid receptacle to a dialysis tubing set is provided, comprising the steps of opening or releasing a clamping mechanism that interfaces with the fluid delivery line, backfiltering dialysate through a dialyzer of the dialysis system, into the dialysis tubing set, and into the fluid receptacle via the fluid delivery line, performing dialysis therapy with the dialysis system including pulling blood into the dialysis tubing set and returning the blood to the patient with the backfiltered dialysate in the fluid receptacle.

In some embodiments, backfiltering dialysate through the dialyzer further comprises controlling a first dialysate pump that is upstream of the dialyzer to have a faster pump speed than a second dialysate pump that is downstream of the dialyzer.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 shows one embodiment of a dialysis system.

FIG. 2 illustrates one embodiment of a water purification system of the dialysis system.

FIG. 3 illustrates one embodiment of a dialysis delivery system of the dialysis system.

FIG. 4 illustrates the connection between a saline source, a blood circuit and a patient.

FIG. 5A illustrates a method of delivering saline with a dialysis system.

FIG. 5B is a trace diagram showing a blood pump flow rate and an arterial line pressure during saline delivery.

FIG. 6A illustrates a method of delivering saline with a dialysis system.

FIG. 6B is a trace diagram showing a blood pump flow rate and an arterial line pressure during saline delivery.

FIGS. 7 and 8 illustrate methods of delivering saline with a dialysis system.

FIGS. 9A-9B are a schematic diagram of one embodiment of a dialysis system with a rinseback configuration.

FIG. 10 is a method and flowchart of using backfiltered dialysate to return blood to a patient after dialysis therapy.

### DETAILED DESCRIPTION

This disclosure describes systems, devices, and methods related to dialysis therapy, including a dialysis system that is simple to use and includes automated features that eliminate or reduce the need for technician involvement during dialysis therapy. In some embodiments, the dialysis system can be a home dialysis system. Embodiments of the dialysis system can include various features that automate and improve the performance, efficiency, and safety of dialysis therapy.

In some embodiments, a dialysis system is described that can provide acute and chronic dialysis therapy to users. The system can include a water purification system configured to prepare water for use in dialysis therapy in real-time using available water sources, and a dialysis delivery system configured to prepare the dialysate for dialysis therapy. The dialysis system can include a disposable cartridge and tubing set for connecting to the user during dialysis therapy to retrieve and deliver blood from the user.

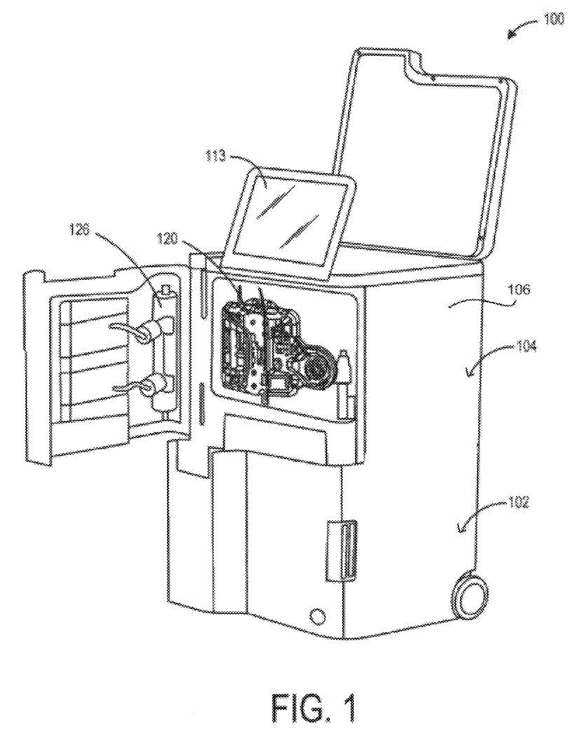

FIG. 1 illustrates one embodiment of a dialysis system 100 configured to provide dialysis treatment to a user in either a clinical or non-clinical setting, such as the user’s home. The dialysis system 100 can comprise a water purification system 102 and a dialysis delivery system 104 disposed within a housing 106. The water purification system 102 can be configured to purify a water source in real-time for dialysis therapy. For example, the water purification system can be connected to a residential water source (e.g., tap water) and prepare pasteurized water in real-time. The pasteurized water can then be used for dialysis therapy (e.g., with the dialysis delivery system) without the need to heat and cool large batched quantities of water typically associated with water purification methodologies.

Dialysis system 100 can also include a cartridge and/or tubing set 120 which can be removably coupled to the housing 106 of the system. The cartridge can include a patient tubing set attached to an organizer, which will be described in more detail below. The cartridge and tubing set, which can be sterile, disposable, one-time use components, are configured to connect to the dialysis system prior to therapy. This connection correctly aligns corresponding components between the cartridge, tubing set, and dialysis system prior to dialysis therapy. For example, the tubing set is automatically associated with one or more pumps (e.g., peristaltic pumps), clamps and sensors for drawing and pumping the user’s blood through the tubing set when the cartridge is coupled to the dialysis system. The tubing set can also be associated with a saline source of the dialysis system for automated priming and air removal prior to therapy. In some embodiments, the saline source can be a source of any generic replacement fluid for dialysis therapy, including but not limited to saline, sterile isotonic fluid, blood, donor plasma, etc. In some embodiments, the cartridge and tubing set can be connected to a dialyzer 126 of the dialysis system. In other embodiments, the cartridge and tubing set can include a built-in dialyzer that is pre-attached to the tubing set. A user or patient can interact with the dialysis system via a user interface 113 including a display.

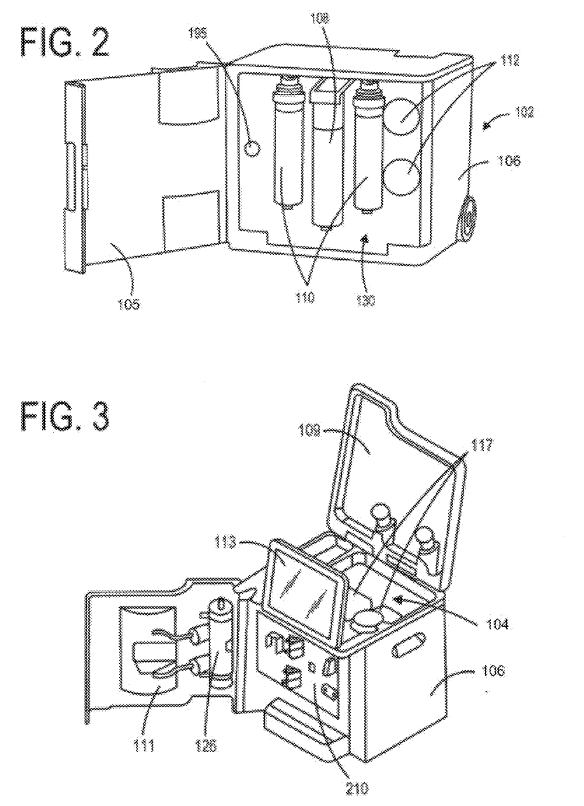

FIGS. 2-3 illustrate the water purification system 102 and the dialysis delivery system 104, respectively, of one embodiment of the dialysis system 100. The two systems are illustrated and described separately for ease of explanation, but it should be understood that both systems can be included in a single housing 106 of the dialysis system. FIG. 2 illustrates one embodiment of the water purification system 102 contained within housing 106 that can include a front door 105 (shown in the open position). The front door 105 can provide access to features associated with the water purification system such as one or more filters, including sediment filter(s) 108, carbon filter(s) 110, and reverse osmosis (RO) filter(s) 112. The filters can be configured to assist in purifying water from a water source (such as tap water) in fluid communication with the water purification system 102. The water purification system can further include heating and cooling elements, including heat exchangers, configured to pasteurize and control fluid temperatures in the system, as will be described in more detail below. The system can optionally include a chlorine sample port 195 to provide samples of the fluid for measuring chlorine content.

In FIG. 3, the dialysis delivery system 104 contained within housing 106 can include an upper lid 109 and front door 111, both shown in the open position. The upper lid 109 can open to allow access to various features of the dialysis system, such as user interface 113 (e.g., a computing device including an electronic controller and a display such as a touch screen) and dialysate containers 117. Front door 111 can open and close to allow access to front panel 210, which can include a variety of features configured to interact with cartridge 120 and its associated tubing set, including alignment and attachment features configured to couple the cartridge 120 to the dialysis system 100. Dialyzer 126 can be mounted in front door 111 or on the front panel, and can include lines or ports connecting the dialyzer to the prepared dialysate as well as to the tubing set of the cartridge.

In some embodiments, the dialysis system 100 can also include a blood pressure cuff to provide for real-time monitoring of user blood pressure. The system (i.e., the electronic controller of the system) can be configured to monitor the blood pressure of the user during dialysis therapy. If the blood pressure of the user drops below a threshold value (e.g., a blood pressure threshold that indicates the user is hypotonic), the system can alert the user with a low blood pressure alarm and the dialysis therapy can be stopped. In the event that the user ignores a configurable number of low blood pressure alarms from the system, the system can be configured to automatically stop the dialysis therapy, at which point the system can inform the user that return of the user’s blood (the blood that remains in the tubing set and dialyzer) back to the user’s body is necessary. For example, the system can be pre-programmed to automatically stop therapy if the user ignores three low blood pressure alarms. In other embodiments, the system can give the user a bolus of saline to bring user fluid levels back up before resuming dialysis therapy. The amount of saline delivered to the patient can be tracked and accounted for during ultrafiltration fluid removal.

The dialysis delivery system 104 of FIG. 3 can be configured to automatically prepare dialysate fluid with purified water supplied by the water purification system 102 of FIG. 2. Furthermore, the dialysis delivery system can de-aerate the purified water, and proportion and mix in acid and bicarbonate concentrates from dialysate containers 117. The resulting dialysate fluid can be passed through one or more ultrafilters (described below) to ensure the dialysate fluid meets certain regulatory limits for microbial and endotoxin contaminants.

Dialysis can be performed in the dialysis delivery system 104 of the dialysis system 100 by passing a user’s blood and dialysate through dialyzer 126. The dialysis system 100 can include an electronic controller configured to manage various flow control devices and features for regulating the flow of dialysate and blood to and from the dialyzer in order to achieve different types of dialysis, including hemodialysis, ultrafiltration, and hemodiafiltration.

The dialysis system can include a connection between a source of saline, or other hemocompatible fluid, to the extracorporeal blood circuit. This fluid can be used for many applications, such as priming the circuit prior to treatment, delivery of boluses to improve hemodynamic stability, delivery of periodic flushes to mitigate circuit clotting, replacement fluid to enable high convective therapies, and a chaser fluid when blood is returned at the end of treatment. Typically, this saline source can be a bag, which is penetrated by a spike attached to a tributary line that tees into the extracorporeal blood circuit. Flow from the saline source can be controlled by clamping and unclamping a physical clamp or electronically controlled pinch valve. It can be advantageous to tee into a part of the blood circuit which is at negative pressure, such as upstream of a blood pump in the circuit, such that the negative pressure will promote flow from the saline source when the line is unclamped.

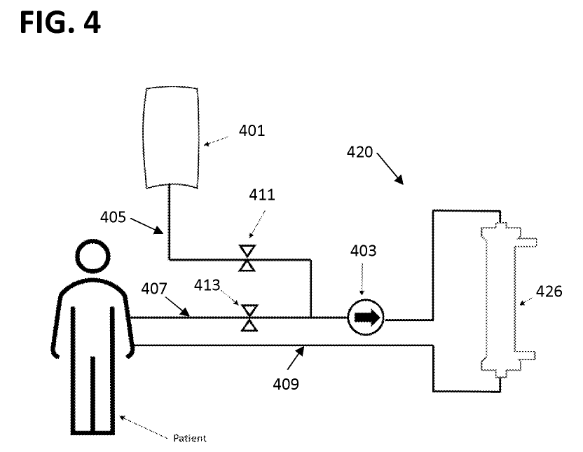

This disclosure provides methods and systems configured to precisely and accurately meter the flow of fluid from the saline source. In one configuration, referring to FIG. 4, a dialysis system (such as the system 100 described above) can include a saline source 401 configured to connect to a tubing set 420 upstream of a blood pump 403. A dialyzer 426 can further be connected to the tubing set, as shown. The tubing set 420 can include a saline line 405, an arterial line 407, and a venous line 409. The tubing set can be configured to interface with one or more pinch clamps such as saline clamp 411 (also referred to as “pre-pump saline pinch valve”) and arterial clamp 413 (also referred to as “arterial pinch valve”). It should be understood that other mechanisms can be used to close/occlude and open/un-occlude the tubing set. Generally, clamps or pinch valves referred to herein include any mechanism that interfaces with the tubing set to turn on or off the flow of fluid through the tubing set.

In one embodiment, the saline claim 411 can be controlled to be unclamped (e.g., the pre-pump saline pinch valve is opened), and the blood pump can be configured to run or operate in a normal operating mode. Although the total output of the blood pump is known, it pulls an indeterminate fraction of its flow from the patient, and a second indeterminate fraction of its flow from the saline source. One technique for metering the amount of saline pulled into the tubing set is to simultaneously occlude the arterial line (with the arterial clamp 413) leading from the patient, and un-occlude the saline line to deliver saline (by opening the saline clamp 411). the pinch valves can be computer controlled, for example, so opening and/or closing the clamps 511 and 413 can be achieved with the electronic controller of the system. By doing so, the entirety of the input and output of the blood pump switches from the patient to the saline source, allowing precise metering of the saline delivered. In normal operation during treatment, the arterial clamp is open to allow flow, and the saline clamp is closed, to prevent saline from entering the circuit.

The tubes that comprise the blood circuit tubing set are typically composed of polymers which exhibit property changes over time, particularly under load. The properties of the saline line tubing, which are held occluded under high force for the majority of treatment, become more important during longer treatments. A typical intermittent hemodialysis (IHD) treatment may last for four hours; however, partial intermittent renal replacement therapy (PIRRT), slow, low-efficiency dialysis (SLED) and continuous renal replacement therapy (CRRT) treatments may last for twenty four hours or more. As tubing set is held in an occluded state by a compressive force, it may take a compression set, or exhibit stress relaxation behavior. When the compressive force is removed to un-occlude the tubing, the degree of material property change may impact how long the tubing takes to open, or whether the tubing opens at all. This is exacerbated if one side of the former occlusion point is under negative pressure (such as upstream of the blood pump), which would tend to urge the walls of the tubing to remain closed against each other.

If the saline line remains occluded despite removal of the compressive force (e.g., by opening the pinch valve(s), then no saline can flow. If the arterial line remains un-occluded when the saline line is attempted to be opened, then no saline is delivered. If the arterial line is occluded while the saline line is attempted to be opened, then a very low negative pressure is created as the pump attempts to pull against two occluded lines. This condition can be detected by the machine, which can lead to unnecessary alarms which disrupt workflow or terminate treatments.

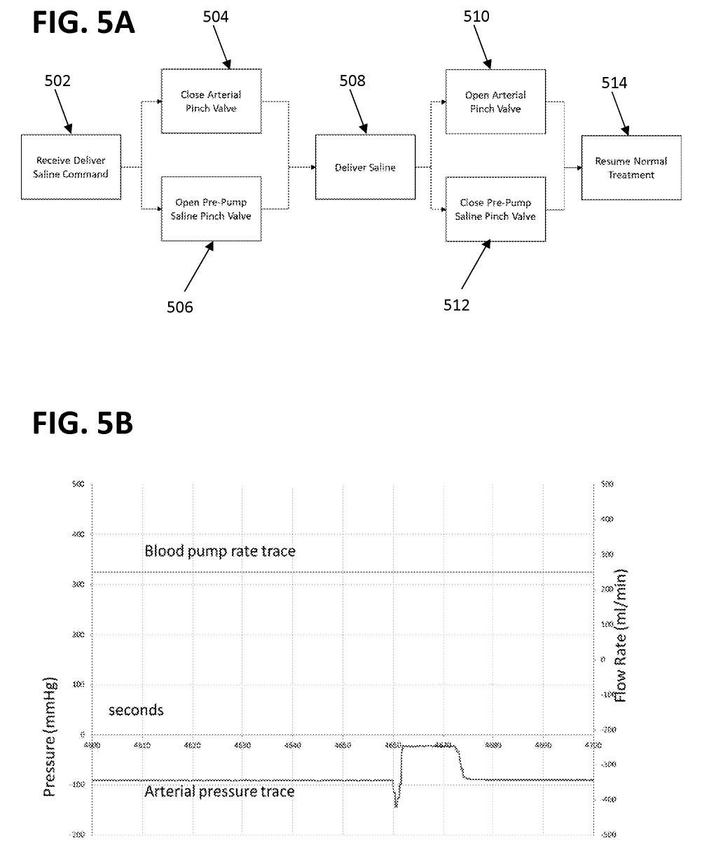

A process flow chart and exemplary trace of the arterial pressure of tubing that has been occluded for 16 hours showing a sharp downward spike is provided in FIGS. 5A and 5B, respectively. For example, referring to FIG. 5A, a method includes, at step 502, receiving a “Deliver Saline” command, which initiates closing the arterial pinch valve and opening the pre-pump saline pinch valve. At steps 504 and 506, the arterial pinch valve can be closed and the pre-pump saline pinch valve can be opened. Next, at step 508, saline is delivered into the blood circuit via the blood pump. When the desired volume of saline has been pulled into the blood circuit, the arterial pinch valve can be opened at step 510, and the pre-pump saline pinch valve can be closed at step 512. Finally, normal treatment is resumed at step 514.

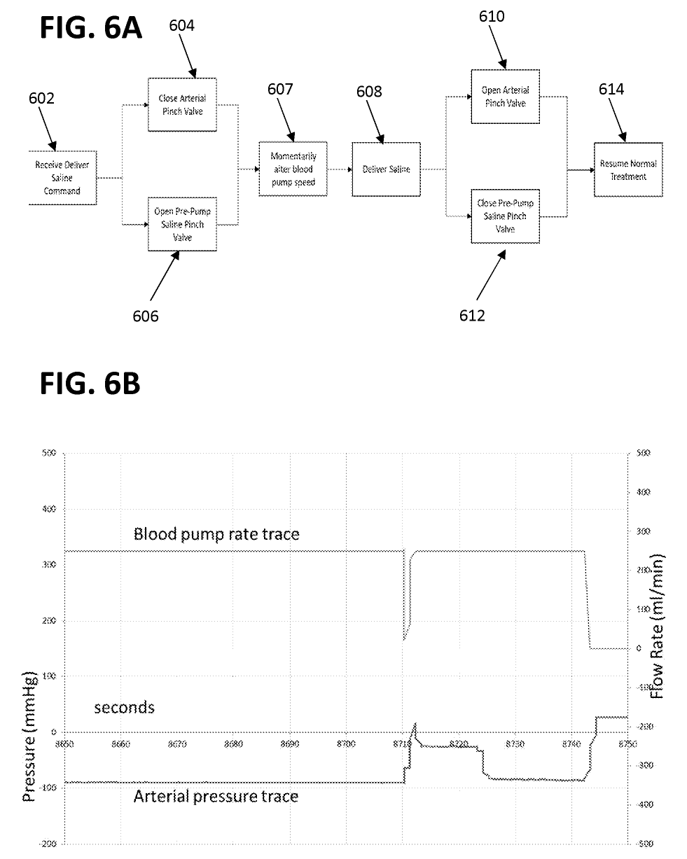

To improve performance of tubing that has been occluded for long periods of time, a novel step is introduced herein wherein after the saline valve is opened and the arterial pinch valve is closed, a step of temporarily modulating the blood pump is introduced. This modulation may comprise slowing, stopping, reversing or pulsing the blood pump. By doing so, the kinetic energy of the fluid flowing through the blood lines must be arrested or absorbed to account for the flow stoppage. This produces a ‘water hammer’ effect that causes a sharp rise in pressure. This rise in pressure propagates to the occlusion point in the saline line, which assists in forcing it open. A modified flow chart and exemplary pressure trace is provided in FIGS. 6A-6B. Referring to FIG. 6A, the novel method of the present disclosure includes, at step 602, receiving a “Deliver Saline” command, which initiates closing the arterial pinch valve at step 604 and opening the pre-pump saline pinch valve at step 606. Next, at step 607, the blood pump speed is altered (e.g., either increased or decreased from the “normal” operating speed or alternatively modulated or pulsed). Next, at step 608, saline is delivered into the blood circuit via the blood pump. When the desired volume of saline has been pulled into the blood circuit, the arterial pinch valve can be opened at step 610, and the pre-pump saline pinch valve can be closed at step 612. Finally, normal treatment is resumed at step 614. Altering the blood pump speed can comprise, for example, temporarily slowing the blood pump speed from a first flow rate (e.g., 320 ml/min) to a second flow rate (e.g., 180ml/min). This eliminates the downward spike shown in FIG. 5B and instead introduces an upward spike in the arterial pressure. In other embodiments, the blood pump speed can be temporarily increased between the first flow rate and the second flow rate. As described above, in some embodiments, the speed of the blood pump can be rapidly changed or “pulsed” during this step.

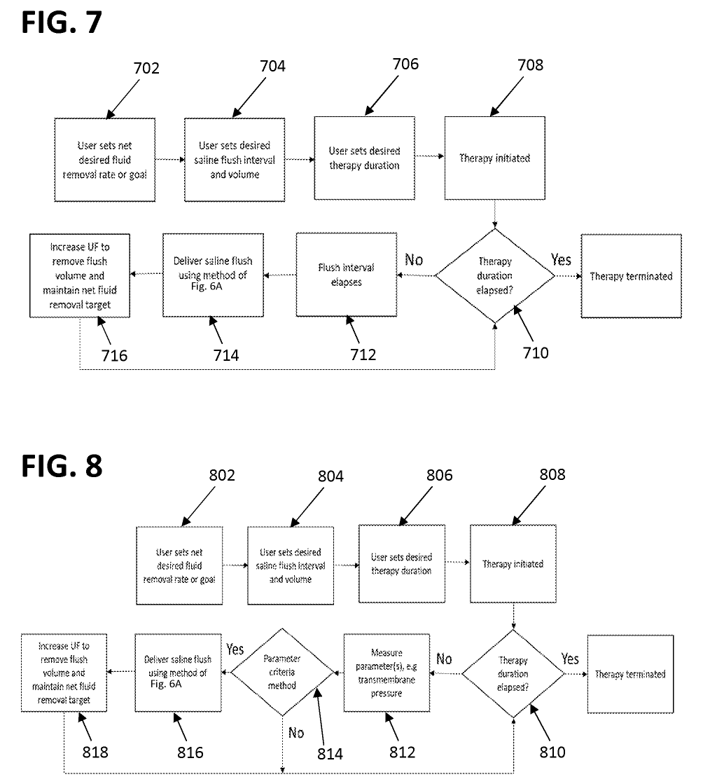

A related aspect of this disclosure is the ability to automatically schedule saline delivery flushes. Referring to the flowchart of FIG. 7, at step 702, a user of the dialysis system can first set a net desired fluid removal rate or goal for the patient. Next, at step 704, the user can specify a desired saline flush interval and volume of saline to be delivered, and a desired therapy duration (step 706), and therapy can be initiated at step 708. Periodically during treatment the flush process of FIG. 6A will occur. This can help to mitigate circuit clotting, allowed for higher fluid removal to enhance convective clearance, or serve as dilution indicator to perform physiological measurements such as blood volume, access recirculation or access flow. For example, still referring to FIG. 7, at step 710 the system can check to see if therapy duration from step 706 has elapsed. If no, then at step 712 the system will check to see if the flush interval from step 704 has elapsed. When this interval elapses, as step 714 the system can deliver saline into the tubing set using the method illustrated in FIG. 6A. At step 716, the system can increase ultrafiltration to remove the saline flush from step 714 from the therapy and maintain the net fluid removal target.

The user may also set a desired interval, prior to the end of treatment, when no saline flushes occur. If the desired frequency and volume of the saline flushes is known, along with the intended treatment time, it is possible to calculate the total saline required. Assuming a known volume per saline container (such as a one liter saline bag), the system would be able to notify the user, prior to treatment, to gather the required number of saline containers to complete the desired saline flush cadence. Alternatively the system may display the required total saline volume, which the user may provide as a single, or multiple larger containers. The system may further determine the unused volume in the last saline container that will be used, (minus volume required for blood return) and recommend increasing volume and/or frequency such that the unused volume is used for additional flushes and not wasted. Alternatively, the overall fluid removal goal can be effectively increased by a volume close to said unused volume, overshooting the fluid removal goal. At the end of treatment, the system can then infuse the unused volume to achieve the original fluid removal goal.

Still referring to FIG. 7, at step 716, the dialysis system may be also configured to automatically increase the fluid removal rate of the patient in order to remove the additional fluid added by these saline flushes over time. Simplistically, this may be adjusted to just remove the excess volume imparted by the saline flush, in order to achieve the desired net fluid removal goal. This decreases the mental burden of the user to calculate and amount of saline infused and manually adjust the fluid removal to account for it. The dilution of the overall blood volume caused by the saline flush can also be used to perform a measurement of the blood volume available. The fluid removal rate may be adjusted based on this measurement as well.

In another embodiment, referring to the flowchart of FIG. 8, the flush interval is not predetermined, but triggered based on the results of measurements of various parameters. Referring to the flowchart of FIG. 8, at step 802, a user of the dialysis system can first set a net desired fluid removal rate or goal for the patient. Next, at step 804, the user can specify a desired saline flush interval and volume of saline to be delivered, and a desired therapy duration (step 806), and therapy can be initiated at step 808. For example, still referring to FIG. 8, at step 810 the system can check to see if therapy duration from step 806 has elapsed. If no, then at step 812 the system can measure parameters associated with the dialysis system, the dialyzer, or dialysis therapy. In one embodiment, the parameter can comprise transmembrane pressure. For example, transmembrane pressure is defined as the difference in the pressure in the dialysate side of the dialyzer, and the blood side of the dialyzer. If clotting within the dialyzer occurs, the resistance to flow between the two compartments increases, and therefore the transmembrane pressure increases. For high-flux dialyzers, there is typically very low transmembrane pressure, even at high ultrafiltration rates, unless there is some degree of clotting. If the transmembrane pressure is detected to meet a certain threshold at step 814, a saline flush can be initiated automatically at step 816 by the dialysis system. At step 818, the ultrafiltration can be increased to remove flush volume and maintain the net fluid removal target during therapy, as discussed above.

Other parameters, such as the slope of the transmembrane pressure curve may be measured or taken into account, which additionally may be used to modulate factors such as the saline flush volume or delivery flow rate. These may additionally be modulated by the user. Other measurements may be considered, for example a ‘scout’ flush of relatively small volume may be released periodically, in order to measure the transit time of the dilution between two sensors disposed in the circuit both pre and post dialyzer. If clotting within the dialyzer is detected, the volume within the fibers decreases, which causes the transit time of the dilution to decrease, relative to a baseline measurement taken earlier, or when there is no clotting. The coagulation cascade also has been reported to induce a change in the conductivity of the blood. These signal can also be used to trigger saline flushes. Alternatively, when any such measurement thresholds are reached, the system may notify the user and recommend a saline flush, which must be confirmed by the user, rather than automatically triggering one.

Further feedback may be built into this measurement-based approach for saline flush control. For example, a saline flush may be initiated once the transmembrane pressure reaches a certain threshold. After the flush, the transmembrane pressure is evaluated again, and another flush is initiated if the measurement is still above the threshold, or a different threshold. These flushes can repeat until the desired value of transmembrane pressure is achieved. The system may be configured to notify the user if a maximum volume or number of flushes has failed to reduce the transmembrane pressure to the desired volume.

As described herein, the added step of modulating blood pump rate can advantageously assist in opening saline lines that may have taken a compression set during long treatments. Furthermore, the dialysis system can be configured to automatically schedule saline flushes and to increase the fluid removal rate to account for excess fluid delivered. In some embodiments, the saline flush interval is dependent on measured parameters from the dialysis system (e.g., measured pressures, flow rates, fluid parameters, etc.).

The methods and systems describe herein provide for fewer nuisance alarms and accurate saline delivery. Additionally, the methods and systems described herein increase user convenience for controlling circuit clotting, or increasing convective clearance and/or something or performing physiological measurements, without the need to manually flush and adjust fluid removal goal.

For circuit clotting, current anticoagulation strategy involve systemic anticoagulant agents such as heparin or citrate, which lead to increased risk of bleeding or severe electrolyte changes (calcium), metabolic alkalosis, or accumulation of the substance if not metabolized effectively by the patient. Using the scheduled saline flushes as described herein overcomes this.

One issue that arises is in the case of machine failure during treatment. Assuming the dialysate fluid is produced in real-time, and is used to return the patient’s blood in contrast to a filled saline bag, such a failure may prevent the patient’s blood from being able to be returned, as the fluid is only produced “on-demand.” The other functions, such as priming of the lines and delivery of fluid boluses, are not as impacted due to only being needed when the machine is functional. Therefore, it desirable to provide a means to return the patient’s blood at the end of treatment that is functionally independent of whether the broader machine systems are still functional, while at the same time taking advantage of logistical simplicity of not requiring a saline bag.

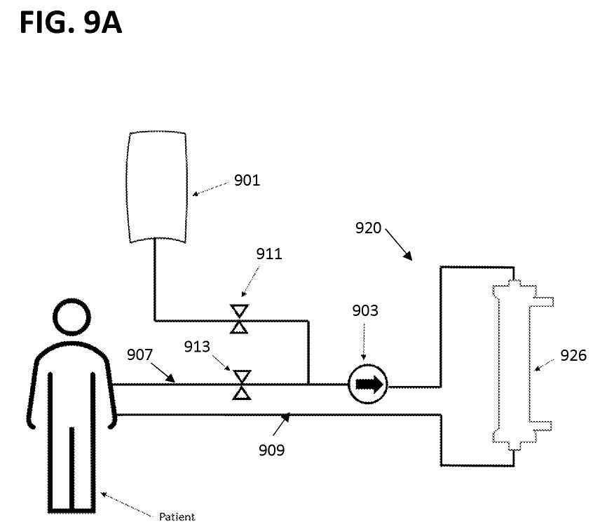

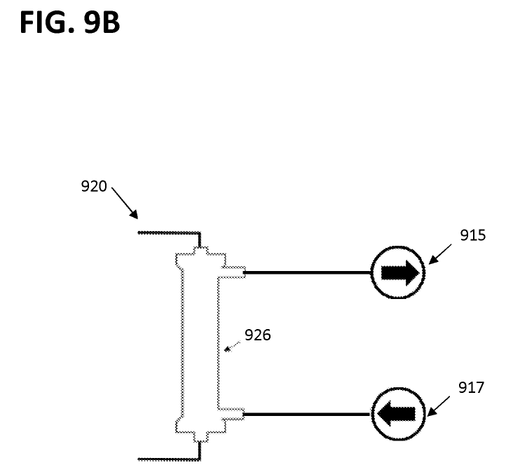

In one configuration, referring to FIG. 9A, a dialysis system (such as the system 100 described above) can include a rinseback fluid source 901 configured to connect to a tubing set 920 upstream of a blood pump 903. A dialyzer 926 can further be connected to the tubing set, as shown. The tubing set 920 can include a saline line 905, an arterial line 907, and a venous line 909. The tubing set can be configured to interface with one or more pinch clamps such as saline clamp 911 (also referred to as “pre-pump saline pinch valve”) and arterial clamp 913 (also referred to as “arterial pinch valve”).

Referring to FIG. 9B, the dialysis system can further include a plurality of dialysate pumps 915 and 917 disposed on a dialysate side of the dialyzer 926, opposite of the tubing set 920 which is on the “blood-side” of the dialyzer. One of the dialysate pumps can be disposed upstream of the dialyzer and one of the dialysate pumps can be disposed downstream of the dialyzer. The pump speeds of the dialysate pumps can be controlled to manage the flow of dialysate through the dialyzer, and to control the ultrafiltration rate during dialysis therapy. For example, to remove fluid from the patient, the downstream pump 915 can be controlled at a faster pump speed than the upstream pump 917.

The rinseback fluid source 901, analogous to a saline source, is connected to the blood circuit, preferentially with automatically-controlled valves, as above. However, unlike in prior solutions, this rinseback fluid source is empty when shipped. In one embodiment, this rinseback fluid source can be pre-connected to the blood tubing set, unlike saline bags which must be accessed through a “spike” connector, which introduces a risk of contamination and/or user injury. In some embodiments, the preferred capacity of this bag is about 500mL, and at least 250mL, which is typically the actual volume of fluid needed for rinseback.

As shown in FIG. 9A, the tubing set is connected to a dialysate circuit through a dialyzer 926, which includes a semi-permeable membrane separating the two compartments which allows filtration of the blood. In a preferred embodiment, dialysate of the system is produced in real-time, and preferentially from decomposited powder or liquid components. The dialysis system can include a dialysate mixing system that includes variable proportioning capabilities. For example during priming, a lower concentration of bicarbonate buffer may be added to the dialysate in order to more closely mimic the composition of saline. In some embodiments, the dialysate circuit is able to both remove fluid from the blood circuit (as is needed to remove excess fluid from the patient in the course of normal treatment), as well as to push fluid into the blood circuit, for priming, boluses, and rinseback.

As part of a priming sequence, the dialysis system is configured to back-filter dialysate through the dialyzer into the blood circuit. To backfilter dialysate through the dialyzer, the upstream pump 917 can be controlled at a faster pump speed than the downstream pump 915. Valves, such as saline valve 911, leading to the rinseback fluid source 901 can be controlled to be open, and the rinseback fluid bag will fill with backfiltered priming dialysate. Once a predetermined amount of fluid is pumped into the rinseback fluid source, the valve 911 can be controlled to close or occlude the saline line, and remain closed until rinseback is ready to start. This way, if the machine’s dialysate circuit fails, it is still possible to use the pre-filled fluid from the rinseback bag to conduct blood return. It is sometimes necessary to discard the initial volume of priming fluid that contacts the dialyzer to flush out sterilization residual chemicals. In this case, at the start of prime, the valve 911 to the rinseback fluid source is closed at the start of prime, and the blood circuit is primed without filling the rinseback bag. Then, the blood circuit priming fluid is discarded, and then replaced with new back-filtered dialysate. At this point, the valve to the rinseback bag may be opened and the bag may be filled with the rinseback dialysate fluid, after the prime discard sequence.

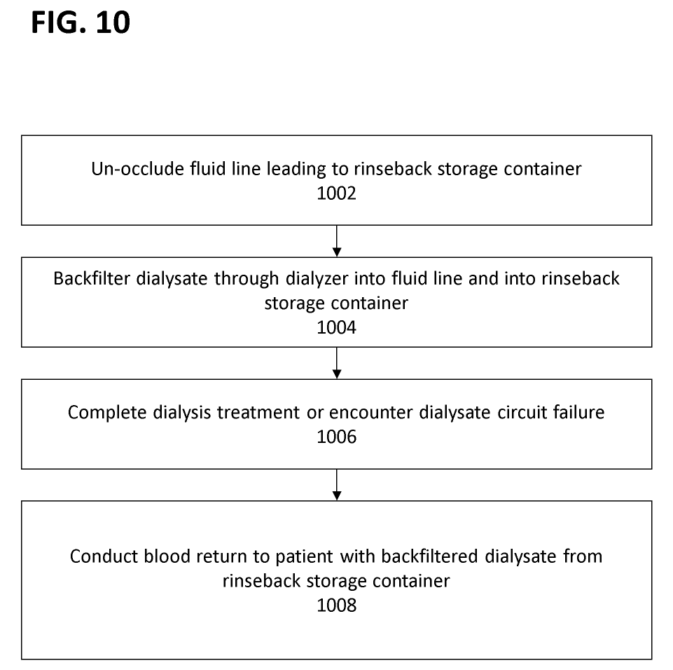

FIG. 10 is a flowchart describing the methodology of using backfiltered dialysate in a rinseback fluid source to return blood to the patient in the event of a dialysate circuit failing during therapy. At step 1002, the fluid line leading to a rinseback storage container (such as rinseback fluid source 901) can be un-occluded or opened. This can be achieved, for example, by opening a pinch valve of the fluid line (such as valve 911 of the saline line). At step 1004, dialysate can be backfiltered through the dialyzer into the fluid line and into the rinseback storage container. At step 1006, the dialysis treatment can be completed. Alternatively, the dialysate circuit can encounter a failure condition. Finally, at step 1008, blood return can be conducted to the patient using the backfiltered dialysate in the rinseback storage container. In some embodiments, in the case of very long treatments (e.g., longer than 24 hours), there may be a need to periodically refresh the fluid to eliminate concerns about microbial growth. In this embodiment, all of the fluid from the fluid source can be pumped out of the source through the dialyzer to drain, and then the same process described above can be initiated to refill the fluid source.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature’s relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/- 0.1% of the stated value (or range of values), +/- 1% of the stated value (or range of values), +/- 2% of the stated value (or range of values), +/- 5% of the stated value (or range of values), +/- 10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A method of improving the performance of dialysis tubing that includes an arterial line and also includes a fluid delivery line connecting a fluid source to the dialysis tubing, comprising the steps of:
occluding the arterial line of the dialysis tubing;
opening or releasing a clamping mechanism that interfaces with the fluid delivery line;
modulating a pump speed of a blood pump that interfaces with the dialysis tubing to increase a fluid pressure within the dialysis tubing; and
delivering fluid into the dialysis tubing from the fluid source through the fluid delivery line.

2. The method of claim 1, wherein modulating the pump speed of the blood pump comprises increasing a flow rate of the blood pump from a first flow rate to a second flow rate.

3. The method of claim 1, wherein modulating the pump speed of the blood pump comprises decreasing a flow rate of the blood pump from a first flow rate to a second flow rate.

4. The method of claim 1, wherein modulating the pump speed of the blood pump comprises pulsing a flow rate of the blood pump.

5. The method of claim 1, wherein modulating the pump speed of the blood pump comprises decreasing a flow rate of the blood pump from approximately 320 ml/min to approximately 180 ml/min.

6. The method of claim 1, further comprising automatically scheduling the occluding, opening, and modulating steps to occur periodically during dialysis therapy.

7. The method of claim 1, wherein a portion of the fluid delivery line remains occluded after opening or releasing the clamping mechanism due to prior extended clamping of the fluid delivery line.

8. The method of claim 1, wherein a portion of the fluid delivery line remains partially occluded after opening or releasing the clamping mechanism due to prior extended clamping of the fluid delivery line.

9. The method of claim 1, further comprising the method steps of:
un-occluding the arterial line;
closing or compressing the clamping mechanism that interfaces with the fluid delivery line; and
initiating dialysis therapy.
